(19) United States
(12) Patent Application Publication
(10) Pub. No.: US 2005/0214379 A1
(43) Pub. Date: Sep. 29, 2005

(54) Encapsulation of chemical compounds in fluorous-core and fluorous-inner-shell micelles formed from semifluorinated-block or fluorinated-block copolymers

(76) Inventors: Sandro Mecozzi, Madison, WI (US); Khanh Hoang, Madison, WI (US)

Correspondence Address: OLYMPIC PATENT WORKS PLLC, P.O. BOX 4277, SEATTLE, WA, 98194-0277, US

(21) Appl. No.: 11/028,948

(22) Filed: Jan. 3, 2005

Related U.S. Application Data
(60) Provisional application No. 60/534,178, filed on Jan. 2, 2004.

(51) Int. Cl. A61K 9/16; A61K 9/50

(52) U.S. Cl. 424/490

(57) ABSTRACT

In one embodiment of the present invention, a block copolymer with a hydrophilic region and a semifluorinated region is synthesized and mixed, below a critical micellar concentration, with a fluorinated drug, and the temperature then lowered, or the block-copolymer concentration then increased, or other solution conditions changed, in order to form fluorous-core, drug-encapsulating micelles. Alternatively, a drug may be taken up by already formed micelles in solution. A suspension of the fluorous-core, fluorinated-drug-encapsulating micelles is injected into the bloodstream to deliver the fluorinated drug to target tissues and organs. In an alternative embodiment of the present invention, a block copolymer with a hydrophilic block, a hydrophobic block, and a semifluorinated block is used to form fluorous-core, drug-encapsulating micelles. In a third embodiment, a block copolymer with a hydrophilic block, a semifluorinated block, and a hydrophobic block is used to form hydrophobic-core, drug-encapsulating micelles. In additional embodiments, block copolymers with various types of blocks are synthesized and employed to form micelles with interior shell and core regions suitable for encapsulating specific target compounds for a variety of purposes.

### CROSS-REFERENCE TO RELATED APPLICATION

This application claims the benefit of Provisional Application No. 60/534,178, filed Jan. 2, 2004.

### TECHNICAL FIELD

The present invention relates to encapsulation of chemical compounds in synthetic vesicles for drug delivery and, in particular, to a drug delivery method and system for encapsulating fluorinated drugs within fluorous-core micelles formed from semifluorinated block copolymers, and for encapsulation of chemical compounds in fluorous-core and fluorous-inner-shell-containing micelles and liposome-like structures.

### BACKGROUND OF THE INVENTION

Delivery of drugs to target tissues and organs within the body is an area of continued research and investigation to which significant effort and expense is currently devoted. In many cases, a drug may be mixed with relatively inert ingredients to form a pill, or inserted into a gelatin capsule, which is ingested to deliver the drug to the bloodstream via the gastrointestinal system. However, this common delivery system is replete with many dependencies, including the drug: (1) passing through the stomach and upper intestine relatively unscathed by the digestive processes; (2) being taken up by the gastrointestinal system and delivered to the bloodstream; (3) traveling through the bloodstream to a target organ or tissue in sufficient concentrations to have a therapeutic effect; (4) being efficiently taken up by the target tissue or target organ to render a therapeutic dose to the tissue or organ; and (5) not producing deleterious side effects in the tissues and organs through which the drug passes from the gastrointestinal system to the target tissue or target organ, and from the target tissue or target organ through catabolic processes to excretion or to anabolic processes by which degradation products of the drug are incorporated into the body. Although many common drugs are delivered in this manner, few drugs are so delivered without problems. Aspirin, for example, can be delivered by ingestion to inhibit cyclooxygenase COX-2 in distant target tissues that synthesize prostaglandins for control of inflammation and fever, but produces significant side effects by inhibiting COX-1 that catalyzes synthesis of prostaglandins that regulate secretion of gastric mucin, leading to irritation and thinning of the stomach lining. As another example, few protein and polypeptide drugs can be administered effectively by ingestion, since proteins and polypeptides are degraded by digestive enzymes.

Alternative drug delivery systems include: (1) inhalation of volatile drugs, drugs that can be dissolved into a volatile carrier, and drugs that can be mixed with a liquid carrier from which an aerosol can be generated; and (2) injection of drugs suspended or dissolved in a carrier liquid directly into the bloodstream. Both delivery systems involve many of the same dependencies as delivery by ingestion, as well as many delivery-system-specific dependencies. For example, injected drugs not only need to be carried effectively by the bloodstream to target tissues and organs, at therapeutic concentrations and for therapeutic durations, but also need to be either nonantigenic or to be chemically encapsulated in order to avoid provoking a potentially fatal immune response. Inhaled drugs need to effectively pass through the membranes of epithelial cells lining the lungs.

Often, effective therapeutic use of drugs requires that not only an effective, primary delivery system be available, but also the availability of at least one alternative delivery system. For example, although a drug may be generally effectively delivered by inhalation, there may be situations in which inhalation is unavailable, such as for unconscious and unstable patients, patients with severe lung congestion, or patients with severely degraded lung capacity or function.

Although drug-delivery systems have been intensively studied, and although many effective systems have been developed for specific drug/target-tissue pairs to supplement the general drug delivery routes of ingestion, injection, and inhalation, there remain many drugs for which effective delivery systems have yet to be discovered, and many drugs that are effectively delivered by a primary delivery system, but for which alternative routes of delivery have yet to be found. For this reason, researchers, pharmaceutical companies, medical professionals, and those needing the benefits of therapeutic drugs have recognized the need for new and alternative drug delivery systems.

### SUMMARY OF THE INVENTION

In one embodiment of the present invention, a block copolymer with a hydrophilic block and a fluorinated or semifluorinated block is synthesized and mixed, below a critical micellar concentration, with a fluorinated drug, and the temperature then lowered, the block-copolymer concentration then increased, or other solution conditions then changed in order to form fluorous-core, drug-encapsulating micelles. Alternatively, a drug may be taken up in solution by already formed micelles.

The fluorinated drug has greater affinity for the fluorous cores of the micelles than for the bulk, aqueous solution in which the fluorous-core micelles form, and therefore may become encapsulated within the fluorous cores of the micelles. In a second embodiment of the present invention, a suspension of the fluorous-core, fluorinated-drug-encapsulating micelles is injected into the bloodstream to deliver the fluorinated drug to target tissues and organs. In a third embodiment of the present invention, a drug with fluorous and hydrophilic components is encapsulated within the fluorous-core micelles at the hydrophilic/semifluorinated block boundary, with the fluorous and hydrophilic components of the drug oriented to be embedded in the semifluorinated core and the hydrophilic shell of the micelles, respectively. In general, different drugs may be encapsulated in different parts of a micelle, depending on the chemical nature of the drugs. Many drugs are quite hydrophobic, and will therefore reside within the inner core of a micelle, or within a fluorinated-polymer-chain shell.

In a fourth embodiment of the present invention, a block copolymer with a hydrophilic block, a hydrophobic block, and a semifluorinated block is synthesized and mixed, below a critical micellar concentration, with a drug that includes hydrophobic and fluorous components, and the temperature then lowered, the block-copolymer concentration then increased, or other solution conditions then changed in order to form fluorous-core, drug-encapsulating micelles. Alternatively, a drug may be taken up by already formed micelles in solution. The drug with hydrophobic and fluorous components may be encapsulated within the fluorous-core micelles at the hydrophobic/semifluorinated block, with the fluorous and hydrophobic components of the drug oriented to be embedded in the semifluorinated core and hydrophobic shell of the micelles, respectively. In alternative embodiments, the drug may be concentrated in different parts of the micelle, depending on the chemical characteristics of the drug, including its hydrophobicity and functional groups that give the drug affinity for different local environments within the micelle. The hydrophobic-inner-shell, fluorous-core micelles can also be used to encapsulate both hydrophobic and fluorinated compounds. In an additional embodiment of the present invention, a copolymer with a hydrophilic block, a fluorinated block, and a hydrophobic, hydrocarbon block is synthesized and used for forming drug-encapsulating micelles. In this embodiment, hydrophobic drugs are encapsulated in the hydrophobic core, and fluorinated drugs may also be encapsulated in the fluorous inner shell. The fluorous inner shell helps to seal the hydrophobic core, as well as lending the greater micelle stability characteristic of fluorous-core micelles and enhancing slow, time-release characteristics of the micelles when used for drug delivery systems. In additional embodiments, block copolymers with various types of blocks are synthesized and employed to form micelles with interior shells and cores suitable for encapsulating specific chemical compounds for a variety of uses, including synthetic, diagnostic, analytic, drug delivery, nanofabrication, and other uses.

### BRIEF DESCRIPTION OF THE DRAWINGS

FIG. 1 illustrates a liposome formed by self aggregation of amphipathic phosphatidylcholine molecules.

FIG. 2 shows the chemical structure of a phosphatidylcholine molecule.

FIG. 3 illustrates a hydrophobic-core micelle formed by self aggregation of amphipathic lysophospholipid molecules.

FIG. 4 shows the chemical structure of the highly fluorinated drug sevoflurane.

FIG. 5 illustrates a first embodiment of the present invention.

FIG. 6 illustrates a hydrophobic-inner-shell, fluorous-core-micelle that represents one embodiment of the present invention.

FIG. 7 shows the chemical structure of a semifluorinated block copolymer that represents one embodiment of the present invention.

FIG. 8 shows the synthetic steps in a synthesis of F8P6 that represents one embodiment of the present invention.

FIG. 9 shows the synthetic steps in a synthesis of 2,2,3,3,4,4,5,5,6,6,7,7,8,8,9,9,10,10,10-heneicosafluoro-1-undecanyl-poly(ethylene glycol) mono-methyl ether that represents one embodiment of the present invention.

### DETAILED DESCRIPTION OF THE INVENTION

Various embodiments of the present invention are directed to drug delivery systems that involve encapsulation of molecules within micelles. Encapsulation of molecules within compartmentalized, hydrophobic and aqueous phases of supramolecular structures is a well-known phenomenon that has been widely exploited for biological research and for drug delivery. Encapsulation of drug molecules is useful for ensuring that the drugs are slowly released within the bloodstream, following injection, in order to provide a therapeutic concentration over a therapeutic time interval. Encapsulation is also useful for shielding a drug from physiological conditions while the encapsulated drug travels to a target tissue or organ. Shielding the drug may prevent the drug from being degraded by catabolic processes, from being bound to unintended targets, from provoking an immune response, and from other consequences ensuing from directly injecting the drug into the bloodstream. Embodiments of the present invention are described in “Aqueous Solubilization of Highly Fluorinated Molecules by Semifluorinated Surfactants,” Langmuir (ACS Journal of Surfaces and Colloids), Volume 20, No. 18, Aug. 31, 2004, pp. 7347-7350, herein incorporated by reference.

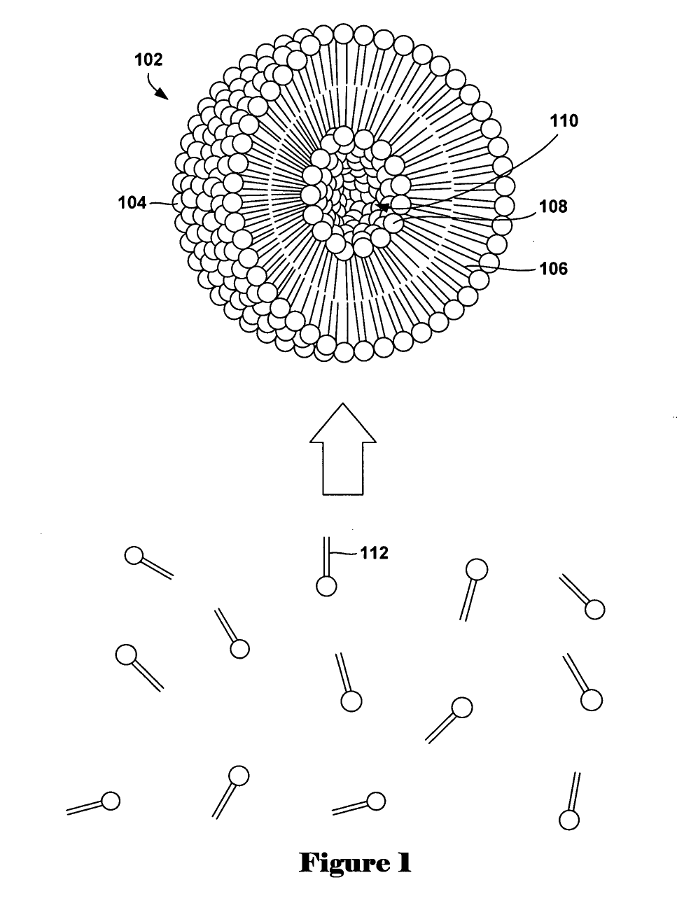

Liposomes are well-known, naturally occurring, as well as synthetically produced, vesicles that can encapsulate water soluble molecules. FIG. 1 illustrates a liposome formed by self aggregation of amphipathic phosphatidylcholine molecules. In FIG. 1, the liposome 102 is shown to be a spherical structure with three distinct shells. An outer shell 104 consists of polar head-group substituents of an outer layer of radially oriented phosphatidylcholine molecules. Liposomes are relatively large structures, with diameters ranging from many tens of nanometers up to a micron or more. An interior shell 106 consists of the hydrophobic lipid substituents of both the outer layer and an inner layer of phosphatidylcholine molecules. An inner shell 108 consists of polar head-group substituents of the inner layer of phosphatidylcholine molecules oriented in radial directions opposite to the orientations of the outer-layer phosphatidylcholine molecules. The interior of the liposome 110 is a generally spherical, aqueous-phase cavity in which water soluble or hydrophilic molecules may be encapsulated by the liposome, in particular, by the relatively thick, hydrophobic interior shell that is relatively impermeable to polar, water-soluble compounds. Liposomes form spontaneously in aqueous media with a sufficiently large concentration of phosphatidylcholine molecules, indicated in FIG. 1 by simple, two-tailed symbols, including symbol 112.

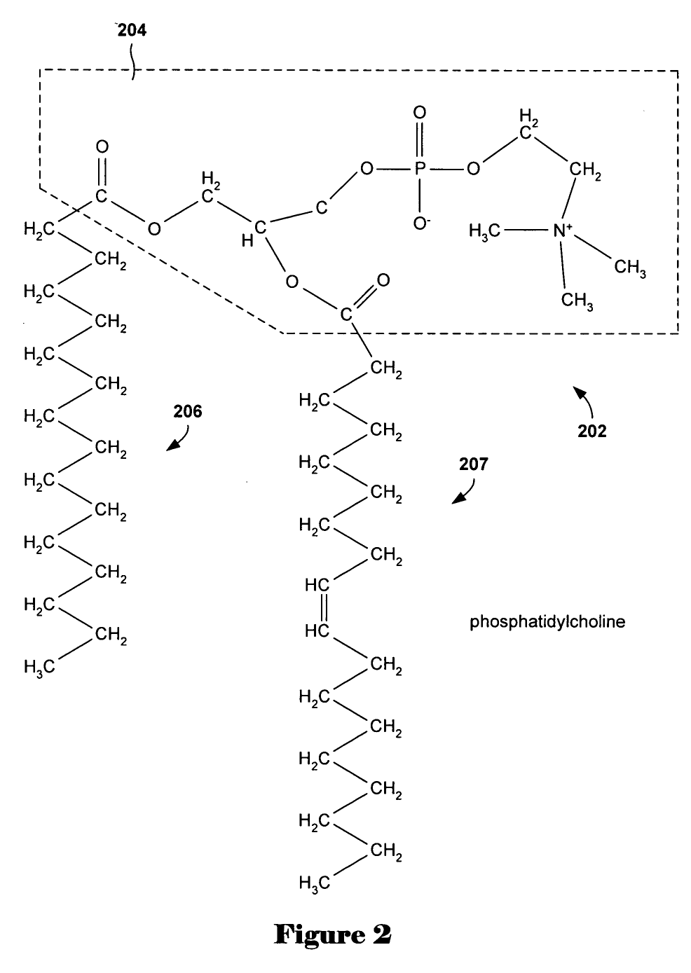

FIG. 2 shows the chemical structure of a phosphatidylcholine molecule. The phosphatidylcholine molecule 202 includes a polar head-group 204 (set off by a dashed polygon in FIG. 2) and two, long, lipid tails 206-207. The liposome structure results from the distinct polar and hydrophobic regions of phosphatidylcholine. Liposomes have been used for encapsulation and delivery of water soluble drugs and for insertion of nucleic acid molecules into the nuclei of cells.

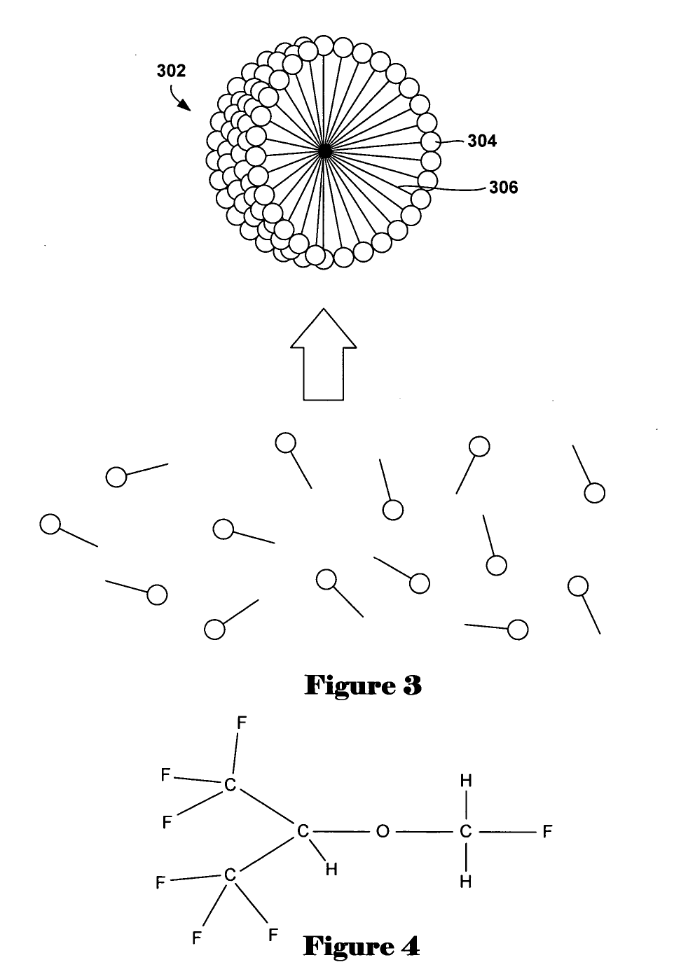

Micelles are somewhat simpler, self-aggregating spherical structures that can be used for drug encapsulation. Micelles are also generally much smaller than liposomes, with diameters of 10-30 nanometers. FIG. 3 illustrates a hydrophobic-core micelle formed by self aggregation of amphipathic lysophospholipid molecules. Lysophospholipids are phospholipids, such as phosphatidylcholine, from which one of the two lipid tails has been removed. A hydrophobic-core micelle 302 is a spherical structure comprising a polar, hydrophilic outer shell 304 and a hydrophobic core 306. Hydrophobic-core micelles therefore resemble liposomes, but lack the inner hydrophilic shell and aqueous-phase cavity. The hydrophobic core 306 does not have a rigid, crystalline structure, but instead is a fluid phase stabilized by hydrophobic and van der Waals interactions. In the hydrophobic core, non-polar molecules are either soluble or at least in thermodynamically favored states with respect to the external aqueous environments, and non-polar molecules can therefore be encapsulated within the hydrophobic core of a micelle as the micelle forms. Hydrophobic-core micelles may be composed of various different types of amphiphilic molecules in addition to lysophospholipids, including block copolymers, detergents, and fatty acids. Hydrophobic-core micelles spontaneously form when the concentration of the particular amphiphilic molecule reaches a critical micellar concentration (“CMC”). Unfortunately, the CMC for many hydrophobic-core micelles is sufficiently large that, when a solution containing suspended, hydrophobic-core micelles is injected into the bloodstream, the concentration of the amphiphilic molecules immediately falls well below the CMC, and the micelles dissipate, releasing encapsulated drug molecules.

While liposomes may, in certain cases, be suitable for encapsulation and delivery of water soluble, polar drugs, and hydrophobic-core micelles may be suitable, in some cases, for encapsulation and delivery of hydrophobic drugs, there are many classes of drugs that do not fall into either category. For example, the pharmaceutical industry is currently developing many new fluorinated drugs, and many fluorinated drugs have been developed and commercialized by the pharmaceutical industry during the past ten years. Highly fluorinated drugs may exhibit both hydrophobic and lipophobic tendencies, and may thus neither be well solvated by, nor show high affinity for, either the internal aqueous cavity of liposomes or the hydrophobic core of hydrophobic-core micelles.

FIG. 4 shows the chemical structure of the highly fluorinated drug sevoflurane. Sevoflurane is a widely used anesthetic, normally administered by inhalation. However, a secondary delivery system for sevoflurane would be advantageous for patients with damaged or congested lungs, for rapidly boosting the level of sevoflurane in already anesthetized patients, for more effectively and controllably administering sevoflurane during anesthesia, for avoiding irritants, such as desflurane, often co-administered with sevoflurane, and for decreasing the amount of sevoflurane administered to a patient in order to induce and maintain anesthesia.

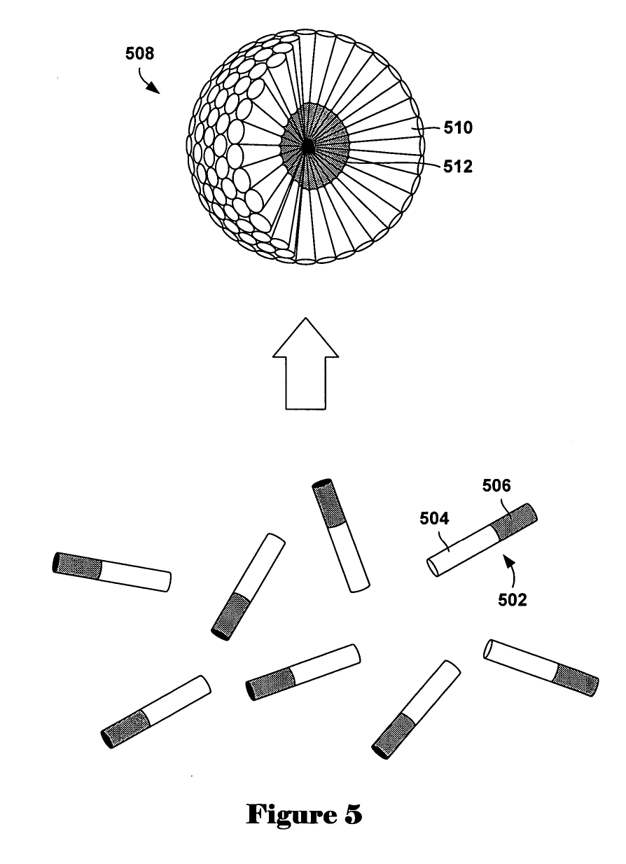

FIG. 5 illustrates a first embodiment of the present invention. In FIG. 5, semifluorinated-block-copolymer molecules, such as semifluorinated-block-copolymer molecule 502, are prepared to contain a hydrophilic block 504 and a semifluorinated, or fluorophilic, block 506. The semifluorinated-block-copolymer molecules self aggregate into stable, fluorous-core micelles 508, with a hydrophilic outer shell 510 and a fluorous core 512. The fluorous core 512 is, like the inner, lipid shell of a liposome, or the hydrophobic core of a hydrophobic-core micelle, a fluid-phase medium. Unlike liposomes and hydrophobic-core micelles, the fluorous core of fluorous-core micelles provide a chemical environment in which highly fluorinated drugs are either soluble or at least in relatively low-energy thermodynamic states with respect to aqueous and hydrophobic environments. When the semifluorinated-block-copolymer is added to a solution containing a fluorinated drug, the fluorinated drug, with higher solubility in the fluorous, fluid-phase medium within the nascent fluorous-core micelles, is encapsulated within the fluorous-core micelles at high efficiency. In addition to fluorous-core micelles providing a fluid core that can solvate fluorinated drugs, fluorous-core micelles exhibit significantly lower CMCs, and are thus less prone to dissipating when injected in a suspension into the bloodstream. The lower CMCs, and greater stability in dilute solutions, arise from the larger van der Waals surfaces of fluorocarbons and lower polarizability of fluorine that together generally give fluorocarbons a greater hydrophobicity than hydrocarbons. Fluorocarbons are lipophobic in addition to being hydrophobic.

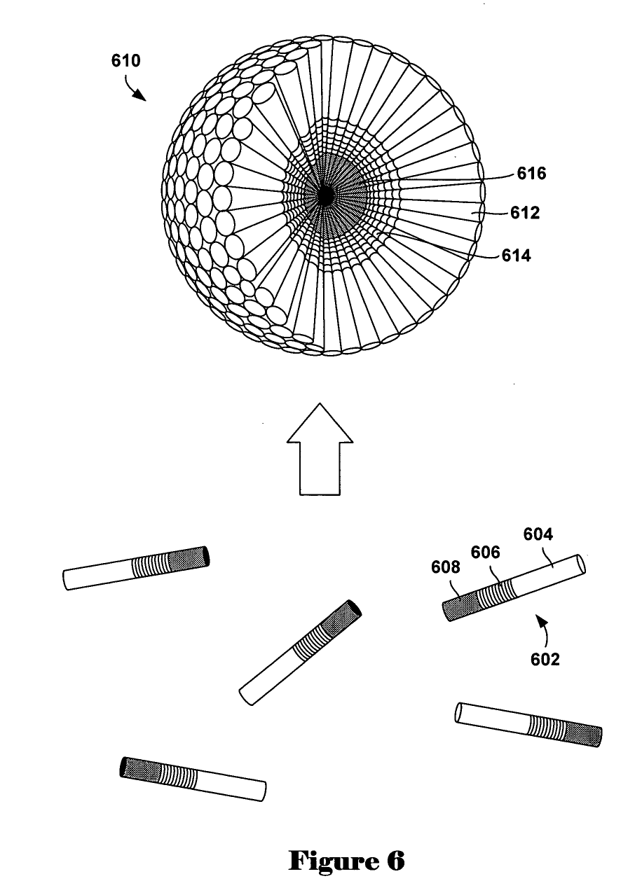

FIG. 6 illustrates a hydrophobic-inner-shell, fluorous-core-micelle that represents one embodiment of the present invention. In FIG. 6, block copolymer molecules, such as block-copolymer molecule 602, are prepared to contain a hydrophilic region 604, a hydrophobic region 606, and a semifluorinated, or fluorophilic, region 608. The block-copolymer molecules self aggregate into stable fluorous-core micelles 610, each with a hydrophilic outer shell 612, a hydrophobic inner shell 614, and a fluorous core 616. The fluorous core 616 is a fluid-phase medium in which highly fluorinated drugs are either soluble or at least in relatively low-energy thermodynamic states. Moreover, nonpolar drugs are soluble in the hydrophobic inner shell 614, as they are in the hydrophobic core of hydrophobic-core micelles. Drugs that include both fluorinated and hydrophobic components or regions may be incorporated at the fluorous-core/hydrophobic-inner-shell boundary, oriented so that the fluorous components are embedded in the fluorous core, and the hydrophobic regions are embedded in the hydrophobic inner shell. The fluorous-core, hydrophobic-shell micelles also exhibit significantly lower CMCs than hydrophobic-core micelles, and are thus less prone to dissipating when injected as a suspension into the bloodstream. The semifluorinated regions of the block copolymers are both lipophobic and hydrophobic, and are thus thermodynamically driven to avoid contact with the polar blocks of other block copolymers, the hydrophobic blocks of other block copolymers, and the aqueous solution in which they form.

In another embodiment, a copolymer with a hydrophilic block, a fluorinated block, and a hydrophobic, hydrocarbon block is synthesized and used for forming drug-encapsulating micelles. In this embodiment, hydrophobic drugs are encapsulated in the hydrophobic core, and fluorinated drugs may also be encapsulated in the fluorous inner shell. The fluorous inner shell helps to seal the hydrophobic core, as well as lending the greater micelle stability characteristic of fluorous-core micelles and enhancing slow, time-release characteristics of the micelles when used for drug delivery systems.

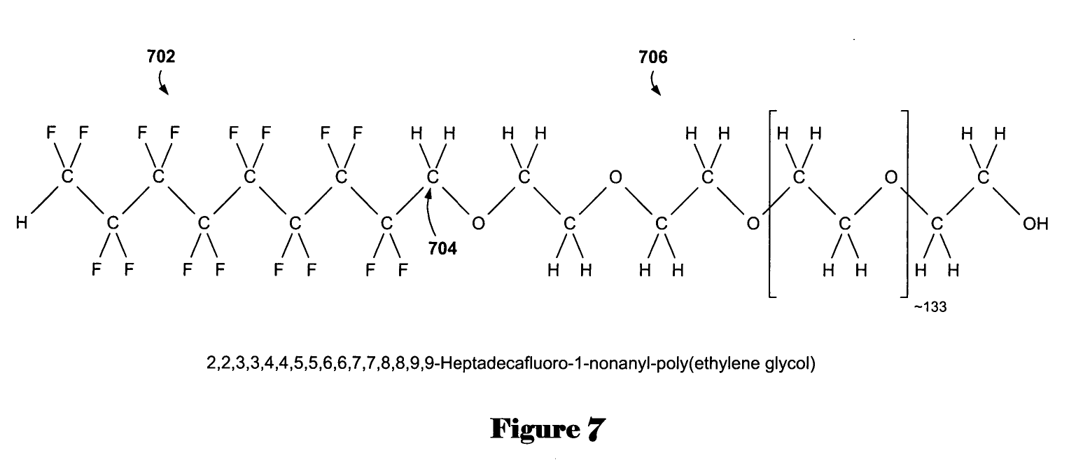

FIG. 7 shows the chemical structure of a semifluorinated block copolymer that represents one embodiment of the present invention. The full chemical name for the semifluorinated block copolymer shown in FIG. 7 is 2,2,3,3,4,4,5,5,6,6,7,7,8,8,9,9-heptadecafluoro-1-nonanyl-poly(ethylene glycol), abbreviated in the following discussion as “F8P6.” F8P6 consists of a highly fluorinated, 8-carbon polymer block linked through a single, bridging alkyl carbon 704 to a hydrophilic polyethylene glycol (“PEG”) polymer block 706 with a number-average molecular weight of 6000 atomic mass units. The PEG polymer block is desirable for the semifluorinated block copolymer because it is relatively non-toxic, highly hydrophilic, and a well-known chemical camouflage agent for shielding antigens from immune-system recognition. F8P6 self aggregates into fluorous-core micelles in water at room temperature with a CMC estimated to be 1 mg/ml. F8P6 micelles are estimated to have a diameter, in water at room temperature, of 13 nm. When sevoflurane is added, at 56° C., to a F8P6 polymer solution, stirred for an hour, and then cooled to room temperature, 15 mM of sevoflurane is fully encapsulated in F8P6 micelles at a F8P6 concentration of 3 mg/ml. In one embodiment, fluorous-core micelles constructed from F8P6 have been determined to each encapsulate more than 300 molecules of sevoflurane, and in an alternative embodiment, 400 molecules of sevoflurane.

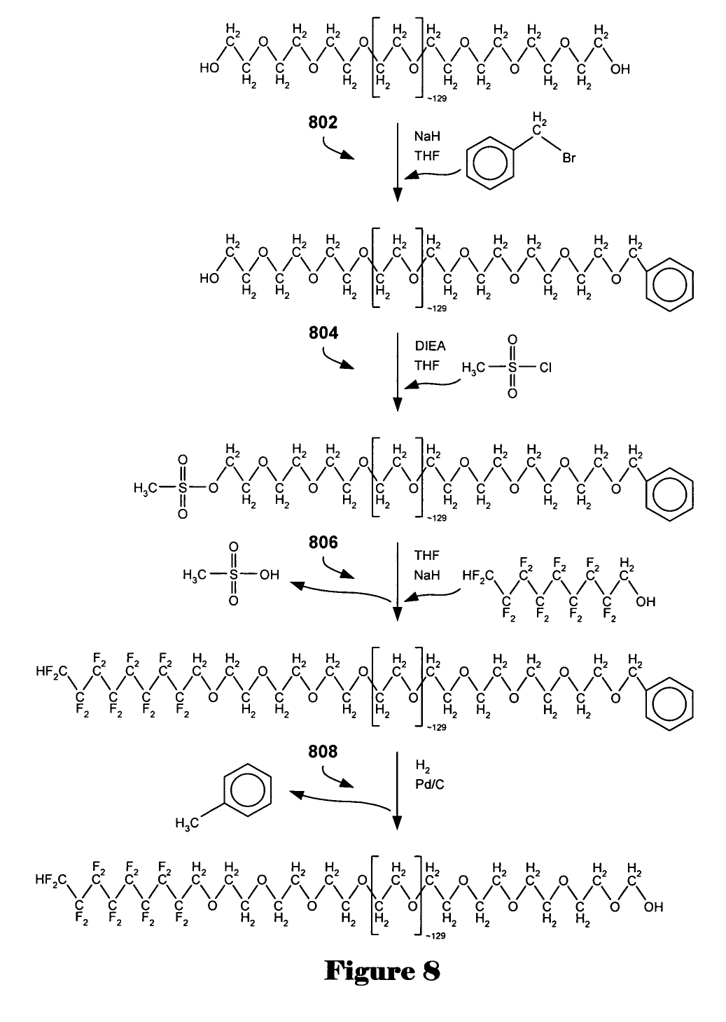

FIG. 8 shows the synthetic steps in a synthesis of F8P6 that represents one embodiment of the present invention. In a first step 802, a 3.3 mmol solution (20 g) of PEG (Mₙ=6000 a.m.u.) in anhydrous tetrahydrofuran (“THF”) is prepared, to which 0.8 g of sodium hydride (“NaH”) is added to a concentration of 10.0 mmol. After stirring for 10 minutes, 0.24 grams of benzyl bromide, C₇H₇Br, is added over the course of 10 minutes by dried syringe to a concentration of 1.4 mmol. The mixture is stirred for 10 hours, and quenched with water. The first step results in protection of one terminal —OH group of the PEG polymer by a benzyl protecting group in a mono-benzyl-protected PEG polymer, along with unwanted di-protected polymer. The THF solvent is partially evaporated, followed by addition of ethyl ether to recrystallize the mono- and di-protected PEG.

In a second step 804, 0.16 g of methanesulfonyl chloride, CH₃SO₂Cl, and 0.2 g of N,N-diisopropylethylamine (“DIEA”) are added to concentrations of 1.4 mmol and 1.5 mmol, respectively, to the benzyl-protected PEG in anhydrous THF in order to mesylate the unprotected terminal —OH group of the mono-benzyl-protected PEG polymer. In an alternative synthesis, tosyl chloride may be added to tosylate the terminal —OH group. The reaction mixture is stirred overnight, and the resulting benzyl-methanesulfonyl poly(ethylene glycol) is recovered, at a 50% yield, by partial evaporation of the THF solvent and recrystallization using ethyl ether.

In a third step 806, 4.8 g of benzyl-methanesulfonyl poly(ethylene glycol) is added to anhydrous THF to a concentration of 0.8 mmol, to which is added 0.5 g of NaH and to which 0.36 g of the semifluorinated compound 2,2,3,3,4,4,5,5,6,6,7,7,8,8,9,9-heptadecafluoro-1-nonanol is added to a concentration of 0.8 mmol in order to join the semifluorinated compound to the mesylated PEG polymer by nucleophilic substitution of the mesyl group. The reaction mixture is then refluxed for 2 days, quenched with water, the THF solvent partially evaporated, and ethyl ether added to recrystallize perfluoroalkyl-benzyl-poly(ethylene glycol).

In a fourth step 808, the benzyl protecting group is removed under H₂ in the presence of 10% activated palladium/carbon, Pd/C, catalyst in 95% absolute ethanol for 10 hours. The mixture is filtered through a Celite® 545 pad to remove Pd/C powder and the ethanol solvent is rota-evaporated. The solid product is dissolved in water, dialyzed for 7 hours inside a Septra/por® membrane with a molecular weight cut-off of 3500 a.m.u., and extracted 5 times with perfluorinated polyethylene ether (FC-72). The five perfluorinated polyethylene ether extractant phases are combined, the solvent rota-evaporated, and the resulting F8P6 polymer is lyophilized to yield powdered F8P6 at a 70% yield for steps 3 and 4. Alternatively, the polymer product can be precipitated with ethyl ether, triturated with hexane and refluxed for 2 hours, suspended in tert-butyl methyl ether, refluxed, and the tert-butyl methyl ether evaporated to produce the pure, solid polymer product.

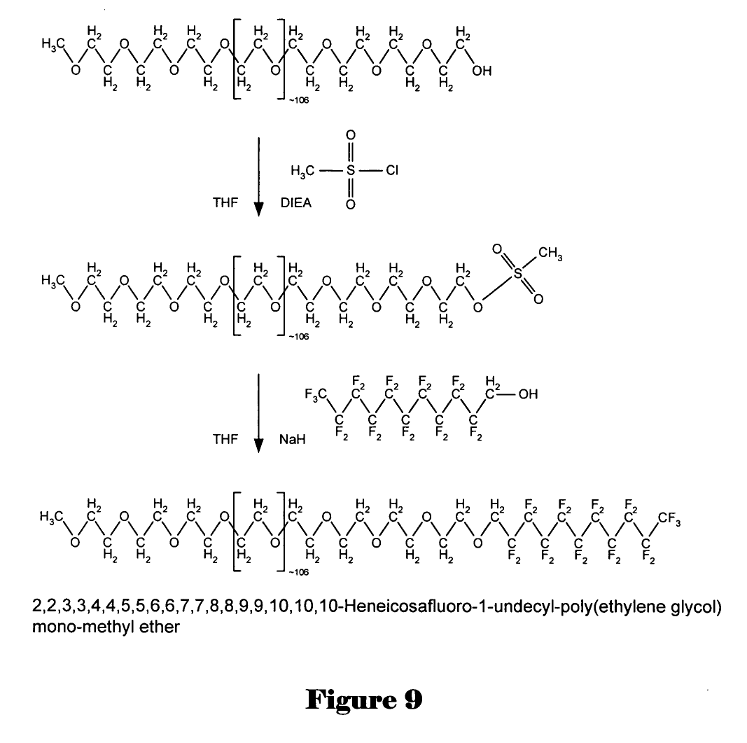

FIG. 9 shows the synthetic steps in a synthesis of 2,2,3,3,4,4,5,5,6,6,7,7,8,8,9,9,10,10,10-heneicosafluoro-1-undecanyl-poly(ethylene glycol) mono-methyl ether that represents one embodiment of the present invention. In a first step 902, a solution of poly(ethylene glycol) mono-methyl ether, 5 equivalents of mesyl chloride, and 10 equivalents of N,N-diisopropylethylamine (“DIEA”) in anhydrous tetrahydrofuran (“THF”) is prepared, which leads to a mesylated poly(ethylene glycol) mono-methyl ether product. In a second step 904, 2 equivalents of 2,2,3,3,4,4,5,5,6,6,7,7,8,8,9,9,10,10,10-heneicosafluoro-1-undecanol and 20 equivalents of sodium hydride (“NaH”) are added to a solution of mesylated poly(ethylene glycol) mono-methyl ether in THF to produce the final product 2,2,3,3,4,4,5,5,6,6,7,7,8,8,9,9,10,10,10-heneicosafluoro-1-undecanyl-poly(ethylene glycol) mono-methyl ether (“HFUPEG”). The HFUPEG product can be precipitated from the solution by adding ethyl ether, and obtained as a solid by vacuum filtration.

Although the present invention has been described in terms of particular embodiments, it is not intended that the invention be limited to those embodiments. Modifications within the spirit of the invention will be apparent to those skilled in the art. For example, although synthesis of the specific block copolymer F86P is described as one embodiment of the present invention, and synthesis of the block copolymer 2,2,3,3,4,4,5,5,6,6,7,7,8,8,9,9,10,10,10-heneicosafluoro-1-undecanyl-poly(ethylene glycol) mono-methyl ether is described as an alternative embodiment of the present invention, a very large number of chemically distinct block copolymers suitable for encapsulation of specific drugs can be devised according to the above-described principles. The disclosed semifluorinated/hydrophilic block copolymers are suitable for encapsulating sevoflurane for injection, but are also useful for encapsulation of a large number of highly fluorinated drugs. The semifluorinated/hydrophobic/hydrophilic-3-block copolymer described above may be suitable for encapsulation of a wide variety of fluorinated and hydrophobic drugs, and drugs containing both fluorinated and hydrophobic regions or component parts. Although synthesis of a specific semifluorinated/hydrophobic/hydrophilic-3-block copolymer is not provided, above, candidate copolymers include F8P6-like molecules in which the bridging alkyl carbon (704 in FIG. 7) is expanded into a hydrocarbon polymer block with 8 or more carbons. Additional, semifluorinated and fluorinated block copolymers similar to F8P6, but with longer and shorter semifluorinated chains, may also be used. For example, a C₁₀F₂₁ or C₆F₁₃ fluorinated block may be used to form fluorous-core micelles with different drug-encapsulation and time-release characteristics. Semi-fluorinated and fluorinated blocks as long as C₂₀F₄₁ can be used to form stable, drug-encapsulating micelles. In alternative embodiments, as discussed above, the order of the regions in the semifluorinated/hydrophobic/hydrophilic-3-block copolymer may be changed to generate micelles with hydrophobic cores and fluorous inner shells. In additional alternative embodiments, the hydrophilic block of the copolymer may be chemically altered or substituted to direct micelles to specific organs or tissues, including adding chemical substituents that are recognized and bound by specific biological receptors, that are preferentially taken up by specific target tissues or organs, or that provoke specific responses, including immune responses, that present a suitable physiological environment for activation or chemical activity of the encapsulated drug. The blocks of the block copolymer used to form micelles may be chemically altered to adjust toxicity, micelle dissipation at appropriate times, solubility of particular drugs within inner shells or cores of micelles, and for other reasons. While F8P6 forms micelles in water at suitable concentrations, different block copolymers may lead to liposome-like structures that include an aqueous cavity enclosed by an inner shell having particular properties useful for specific drug delivery systems. In addition, various other types of supramolecular structures comprising polymers with fluorinated or semifluorinated blocks may stably form in solution, and may be used for encapsulating and transporting pharmaceuticals within biological fluids. Additional supramolecular structures include tube-like structures, vesicles, folded-sheet-like structures, bilayers, films, and complex irregular structures. Embodiments of the present invention depend on the stabilization of pharmaceuticals within fluorinated or semifluorinated regions of stable supramolecular structures, rather than on the particular form of the structures. Although injection of fluorous-core micelles and other fluorous-phase-containing supramolecular structures is one possible method for administering drugs encapsulated in the fluorous-core micelles and other fluorous-phase-containing supramolecular structures, other methods of introducing fluorous-core micelles and other fluorous-phase-containing supramolecular structures into a patient or animal may be used, including introducing the fluorous-core micelles and other fluorous-phase-containing supramolecular structures into a biological or synthetic fluid external to the patient, such as during dialysis, by absorption of the fluorous-core micelles and other fluorous-phase-containing supramolecular structures through skin or membranes, and by other means. Although the above described embodiments are directed to drug delivery, alternative embodiments of fluorous-core micelles may be directed to intermediate micelle nanostructures useful in drug synthesis, micelles useful for analytic and diagnostic purposes, micelles useful for sequestering fluorinated and other types of molecules for materials recovery, pollution abatement, and for other purposes. Fluorous-core micelles may also find use in nanotechnology, for ordering and placing fluorinated small-molecules at designated places within nanofabricated devices.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A fluorophilic-chemical-encapsulation system comprising:
a fluorophilic chemical compound; and
a supramolecular structure comprising a number of block-copolymer molecules, each block-copolymer molecule containing at least one of
a fluorinated block, and
a semifluorinated block.

2. The fluorophilic-chemical-encapsulation system of claim 1 wherein the fluorophilic chemical compound contains at least one fluorine atom.

3. The fluorophilic-chemical-encapsulation system of claim 1 wherein the fluorophilic chemical compound is a drug that contains at least one fluorine atom.

4. The fluorophilic-chemical-encapsulation system of claim 3 wherein the drug is sevoflurane.

5. The fluorophilic-chemical-encapsulation system of claim 1 wherein the supramolecular structure comprising a number of block-copolymer molecules is a fluorous-core micelle comprising one of:
polyethylene-glycol/semifluorinated-alkane block-copolymer molecules; and
polyethylene-glycol/fluorinated-alkane block-copolymer molecules.

6. The fluorophilic-chemical-encapsulation system of claim 5 wherein the block-copolymer molecules each includes a polyethylene glycol block containing between 20 and 300 ethoxy monomers.

7. The fluorophilic-chemical-encapsulation system of claim 5 wherein the block-copolymer molecules each include one of a semifluorinated alkane and fluorinated alkane having between 4 and 70 carbon atoms.

8. The fluorophilic-chemical-encapsulation system of claim 5 wherein the block-copolymer molecules are one of:
2,2,3,3,4,4,5,5,6,6,7,7,8,8,9,9,10,10,10-heneicosafluoro-1-undecanyl-poly(ethylene glycol) mono-methyl ether; and
2,2,3,3,4,4,5,5,6,6,7,7,8,8,9,9-heptadecafluoro-1-nonanyl-poly(ethylene glycol).

9. The fluorophilic-chemical-encapsulation system of claim 1 wherein the supramolecular structure comprising a number of block-copolymer molecules is a fluorous-core micelle containing block-copolymer molecules each having at least one hydrophilic block, one hydrophobic block, and one fluorinated or semifluorinated block.

10. The fluorophilic-chemical-encapsulation system of claim 1 wherein the supramolecular structure comprising a number of block-copolymer molecules is a hydrophobic-core micelle containing block-copolymer molecules each having at least one hydrophilic block, one fluorinated or semifluorinated block, and one hydrophobic block.

11. The fluorophilic-chemical-encapsulation system of claim 1 wherein the supramolecular structure comprising a number of block-copolymer molecules contains a fluorous-phase region and is one of:
a micelle;
a tube-like supramolecular structure;
a vesicle;
a folded-sheet supramolecular structure;
a bilayer;
a regular film; and
a complex irregular structure.

12. A method for administering a fluorophilic drug, the method comprising:
encapsulating the fluorophilic drug into a supramolecular structure comprising a number of block-copolymer molecules, each block-copolymer molecule containing at least one of
a fluorinated block, and
a semifluorinated block; and
introducing the fluorophilic drug into a patient.

13. The method of claim 12 wherein the fluorophilic drug is introduced into the patient by one of:
injection;
dialysis; and
absorption.

14. The method of claim 12 wherein the fluorophilic drug contains at least one fluorine atom.

15. The method of claim 12 wherein the fluorophilic drug is sevoflurane.

16. The method of claim 12 wherein the supramolecular structure comprising a number of block-copolymer molecules is a fluorous-core micelle comprising one of:
polyethylene-glycol/semifluorinated-alkane block-copolymer molecules; and
polyethylene-glycol/fluorinated-alkane block-copolymer molecules.

17. The method of claim 16 wherein the block-copolymer molecules each includes a polyethylene glycol block containing between 20 and 300 ethoxy monomers.

18. The method of claim 16 wherein the block-copolymer molecules each include one of a semifluorinated alkane and fluorinated alkane having between 4 and 30 carbon atoms.

19. The method of claim 16 wherein the block-copolymer molecules are one of:
2,2,3,3,4,4,5,5,6,6,7,7,8,8,9,9,10,10,10-heneicosafluoro-1-undecanyl-poly(ethylene glycol) mono-methyl ether; and
2,2,3,3,4,4,5,5,6,6,7,7,8,8,9,9-heptadecafluoro-1-nonanyl-poly(ethylene glycol).

20. The method of claim 12 wherein the supramolecular structure comprising a number of block-copolymer molecules is a fluorous-core micelle containing block-copolymer molecules each having at least one hydrophilic block, one hydrophobic block, and one fluorinated or semifluorinated block.

21. The method of claim 12 wherein the supramolecular structure comprising a number of block-copolymer molecules is a hydrophobic-core micelle containing block-copolymer molecules each having at least one hydrophilic block, one fluorinated or semifluorinated block, and one hydrophobic block.

22. The chemical-encapsulation system of claim 12 wherein the supramolecular structure comprising a number of block-copolymer molecules contains a fluorous-phase region and is one of:
a micelle;
a tube-like supramolecular structure;
a vesicle;
a folded-sheet supramolecular structure;
a bilayer;
a regular film; and
a complex irregular structure.

23. A fluorous-phase-contining micelle component compound comprising:
a polyethylene-glycol block; and
a fluorine-substituted alkane block covalently linked to the polyethylene-glycol block.

24. The fluorous-phase-contining micelle component compound of claim 23 wherein the polyethylene-glycol block includes between 20 and 300 ethoxy monomers.

25. The fluorous-phase-contining micelle component compound of claim 23 wherein the polyethylene-glycol block terminates in a methoxy group.

26. The fluorous-phase-contining micelle component compound of claim 23 wherein the fluorine-substituted alkane block is a semifluorinated alkane having between 4 and 70 carbon atoms.

27. The fluorous-phase-contining micelle component compound of claim 23 wherein the fluorine-substituted alkane block is a fluorinated alkane having between 4 and 30 carbon atoms.

28. The fluorous-phase-contining micelle component compound of claim 23 further comprising one of:
2,2,3,3,4,4,5,5,6,6,7,7,8,8,9,9,10,10,10-heneicosafluoro-1-undecanyl-poly(ethylene glycol) mono-methyl ether; and
2,2,3,3,4,4,5,5,6,6,7,7,8,8,9,9-heptadecafluoro-1-nonanyl-poly(ethylene glycol).
